(19) United States
(12) Patent Application Publication
(10) Pub. No.: US 2019/0380579 A1
(43) Pub. Date: Dec. 19, 2019

(54) BIOLOGICAL INFORMATION MEASURING APPARATUS AND METHOD AND PROGRAM USING THE SAME

(71) Applicants: OMRON CORPORATION, Kyoto (JP); OMRON HEALTHCARE CO., LTD., Kyoto (JP)

(72) Inventors: Tsuyoshi KITAGAWA, Kyoto (JP); Shingo YAMASHITA, Kyoto (JP)

(21) Appl. No.: 16/553,558

(22) Filed: Aug. 28, 2019

Related U.S. Application Data
(63) Continuation of application No. PCT/JP2018/009563, filed on Mar. 12, 2018.

(30) Foreign Application Priority Data
Mar. 15, 2017 (JP) 2017-050580

(51) Int. Cl. A61B 5/00; A61B 5/021

(52) U.S. Cl. CPC A61B 5/0004; A61B 5/02108; A61B 5/6824; A61B 2560/0271; A61B 2560/0214; A61B 2560/0276; A61B 2560/0223

(57) ABSTRACT

According to one embodiment, a biological information measuring apparatus including a sensing apparatus and a calibration device. The calibration device includes a measuring device and a transmitter. The sensing apparatus includes a detector, a receiver, and a calculator. The measuring device intermittently measures first biological information. The transmitter transmits data including the first biological information to the sensing apparatus. The detector detects a pulse wave continuously in time. The receiver receives the data from the calibration device. The calculator calibrates the pulse wave based on the first biological information, and calculates second biological information based on the pulse wave.

### CROSS-REFERENCE TO RELATED APPLICATIONS

This application is a Continuation Application of PCT Application No. PCT/JP2018/009563, filed Mar. 12, 2018 and based upon and claiming the benefit of priority from Japanese Patent Application No. 2017-050580, filed Mar. 15, 2017, the entire contents of all of which are incorporated herein by reference.

### FIELD

The present invention relates to a biological information measuring apparatus for continuously measuring biological information, and a method and a program using the same.

### BACKGROUND

With the advances in sensor technology that have allowed for an environment where high-performance sensors are readily available, application of biological information to treatment, for early detection of abnormalities in the body, has been increasingly gaining medical importance.

A biological information measuring apparatus is known that is capable of measuring biological information, such as the pulse and the blood pressure, using information detected by a pressure sensor that is in direct contact with a biological site, through which an artery, such as the radial artery at the wrist, passes (see, for example, Jpn. Pat. Appln. KOKAI Publication No. 2004-113368).

The blood pressure measuring apparatus described in Jpn. Pat. Appln. KOKAI Publication No. 2004-113368 uses a cuff to calculate the blood pressure value at a biological site different from the site with which the pressure sensor is to be in contact, and generates calibration data from the calculated blood pressure value. By calibrating the pressure pulse wave detected by the pressure sensor using the generated calibration data, the blood pressure value is calculated beat by beat.

However, the blood pressure measuring apparatus described in Jpn. Pat. Appln. KOKAI Publication No. 2004-113368 is large in scale, making it difficult to improve the precision in measurement. Moreover, such a blood pressure measuring apparatus is intended to be operated in a limited environment by a specific person, making it difficult for use in routine care or at home. Furthermore, such a blood pressure measuring apparatus inconveniently requires a large amount of tubing and cabling, making it impractical for use on a daily basis or during sleep.

### SUMMARY

According to a first aspect of the present invention, a biological information measuring apparatus comprises a sensing apparatus and a calibration device, the calibration device including: a measuring device that intermittently measures first biological information; and a transmitter that transmits data including the first biological information to the sensing apparatus, the sensing apparatus including: a detector that detects a pulse wave continuously in time; a receiver that receives the data from the calibration device; and a calculator that calibrates the pulse wave based on the first biological information, and calculates second biological information based on the pulse wave.

According to a second aspect of the present invention, the sensing apparatus further includes an instruction transmitter that transmits an instruction to measure the first biological information to the calibration device.

According to a third aspect of the present invention, the detector is disposed on a wrist of a living body, and the measuring unit is disposed closer to an upper arm than the detector.

According to a fourth aspect of the present invention, the detector and the measuring unit are provided at an identical site.

According to a fifth aspect of the present invention, the calibration device further includes: an electric power source unit that supplies electric power to internal device portions; and a monitor that monitors a battery capacity of the electric power source unit; the transmitter transmits capacity data including the battery capacity to the sensing apparatus upon completion of measurement by the measuring device or upon activation of the calibration device, the receiver receives the capacity data, and the sensing apparatus further includes: a capacity-determining unit that determines, based on the capacity data, whether or not the battery capacity has decreased to a level at which the pulse wave cannot be calibrated.

According to a sixth aspect of the present invention, a prompter that prompts charging or replacement of the electric power source unit, upon determining that the capacity-determining unit cannot perform calibration, is further provided.

According to a seventh aspect of the present invention, the calibration device further includes a counting unit that counts a number of measurements performed by the measuring unit; the transmitter transmits number data containing the number of measurements to the sensing apparatus upon completion of measurement by the measuring device or upon activation of the calibration device, the receiver receives the number data, and the sensing apparatus further includes: a number-determining unit that determines, based on the number data, whether or not the number of measurements performed has exceeded a certain number of times of usage.

According to an eighth aspect of the present invention, a prompter that prompts replacement of the calibration device, if the number of measurements performed has exceeded a certain number of times of usage, is further provided.

According to a ninth aspect of the present invention, an acquisition unit, which acquires a first blood pressure value included in the first biological information and a second blood pressure value included in second biological information at a time of day which is earlier, by a certain length of time, than a time of day when the measuring unit commences measurement, and a failure-determining unit, which determines that a failure is likely to be occurring in the sensing apparatus if a difference between the first blood pressure value and the second blood pressure value is equal to or greater than a threshold value, are further provided.

According to a tenth aspect of the present invention, an acquisition unit, which acquires a first blood pressure value included in the first biological information, and a mean blood pressure value of a second blood pressure value included in second biological information during a certain period of time which is earlier, by a certain length of time, than a time of day when the measuring unit commences measurement, and a failure-determining unit, which determines that a failure is likely to be occurring in the sensing apparatus if a difference between the first blood pressure value and the mean blood pressure value is equal to or greater than a threshold value, are further provided.

According to an eleventh aspect of the present invention, an acquisition unit, which acquires a first blood pressure value included in the first biological information, and a second blood pressure value included in second biological information at a time of day which is earlier, by a certain length of time, than a time of day of the commencement of measurement of the first blood pressure value, and further acquires a third blood pressure value measured by the measuring unit at a time of day different from the time of day of measurement of the first blood pressure value, and a fourth blood pressure value included in second biological information at a time of day which is earlier, by a certain length of time, than the time of day when measurement of the third blood pressure value has been commenced, and a failure-determining unit, which determines that a failure is likely to be occurring in the sensing apparatus if a difference between the second blood pressure value and the fourth blood pressure value is greater than a difference between the first blood pressure value and the third blood pressure value, and if the difference between the second blood pressure value and the fourth blood pressure value exceeds a threshold value, are further provided.

According to a twelfth aspect of the present invention, the measuring unit measures the first biological information with higher precision than second biological information obtained from the detector.

According to a thirteenth aspect of the present invention, the detector detects the pulse wave beat by beat, and the first biological information and the second biological information are blood pressures.

According to the first aspect of the present invention, the calibration device intermittently measures first biological information, and transmits data including the first biological information to the sensing apparatus. The sensing apparatus includes a detector that detects a pulse wave continuously in time, a receiver that receives data from the calibration device, and a calculator that calibrates the pulse wave based on the first biological information, and calculates second biological information based on the pulse wave. In addition, the sensing apparatus is separated from the calibration device. Accordingly, the sensing apparatus is made compact, and the sensor can be placed at a position where the pulse wave can be acquired more reliably. Since the pulse wave is calibrated based on biological information measured by the measuring unit, it is possible to calculate high-precision biological information from the pulse wave, thus allowing the user to easily obtain high-precision biological information. In addition, since the measuring device performs measurement only intermittently, the period of time during which the measuring unit interferes with the user is reduced. Moreover, since the calibration device is independently provided, the calibration device can be mounted at a position appropriate for calibration with ease, regardless of the disposition of the sensing apparatus.

According to the second aspect of the present invention, the sensing apparatus transmits an instruction to the calibration device to perform calibration, thereby calibrating detection of the pulse wave at the sensing apparatus. For example, the sensing apparatus is capable of instructing the calibration device to perform detection for calibration, based on the result of detection by the detector.

According to the third aspect of the present invention, the detector is disposed on a wrist of a living body, and the measuring unit is disposed closer to an upper arm than the detector. This ensures detection of the pulse wave at the wrist.

According to the fourth aspect of the present invention, the detector and the calculator are provided at the identical site (for example, the left wrist or right wrist). Thus, the biological information can be acquired from substantially identical sites.

According to the fifth aspect of the present invention, the calibration device further includes: an electric power source unit that supplies electric power to internal device portions; and a monitor that monitors a battery capacity of the electric power source unit; the transmitter transmits capacity data including the battery capacity to the sensing apparatus upon completion of measurement by the measuring device or upon activation of the calibration device, the receiver receives the capacity data, and the sensing apparatus determines, based on the capacity data, whether or not the battery capacity has decreased to a level at which the pulse wave cannot be calibrated, by monitoring the battery capacity of the electric power source unit. This prevents the situation in which accurate calibration cannot be performed due to battery outage in the calibration device during continuous measurement, and allows calibration to be constantly performed with a normal calibration value.

According to the sixth aspect of the present invention, the prompter prompts charging or replacement of the electric power source unit if the determining unit determines that calibration cannot be performed. This allows the user to be ready to use the calibration device any time.

According to the seventh aspect of the present invention, the calibration device-counts a number of measurements performed by the measuring unit, the transmitter transmits, to the sensing apparatus, number data containing the number of measurements performed upon completion of measurement by the measuring device or upon activation of the calibration device, the receiver receives the number data, and the sensing apparatus determines, based on the number data, whether or not the number of measurements performed has exceeded a certain number of times of usage. This prevents the situation in which calibration cannot be performed due to the calibration device reaching the end of its life during, for example, continuous measurement, and allows calibration to be constantly performed with a normal calibration value.

According to the eighth aspect of the present invention, since the prompter prompts replacement of the calibration device if the number of calibrations performed has exceeded a certain number of times of usage. This allows the user to constantly monitor the calibration device to see whether or not the lifespan is nearing its end.

According to the ninth aspect of the present invention, a first blood pressure value, included in the first biological information, and a second blood pressure value, included in second biological information at a time of day which is earlier, by a certain length of time, than a time of day when the measuring unit commences measurement, are acquired. If a difference between the first blood pressure value and the second blood pressure value is equal to or greater than a threshold value, it is determined that a failure is likely to be occurring in the sensing apparatus, and a notification is made that a failure is likely to be occurring in the sensing apparatus. Accordingly, it is possible to detect a failure in the sensing apparatus at an early stage, thus further increasing the period of time during which biological information obtained based on the pulse wave from the detector can be measured with high precision.

According to the tenth aspect of the present invention, a first blood pressure value, included in the first biological information, and a mean blood pressure value of a second blood pressure value, included in second biological information during a certain period of time which is earlier, by a certain length of time, than a time of day when the measuring unit commences measurement, are acquired. If a difference between the first blood pressure value and the mean blood pressure value is equal to or greater than a threshold value, it is determined that a failure is likely to be occurring in the sensing apparatus. Accordingly, it is possible to detect a failure in the sensing apparatus at an early stage, thus further increasing the period of time during which biological information obtained based on the pulse wave from the detector can be measured with high precision.

According to the eleventh aspect of the present invention, a first blood pressure value, included in the first biological information, and a second blood pressure value, included in second biological information at a time of day which is earlier, by a certain length of time, than a time of day when measurement of the first blood pressure value has been commenced, are acquired, and a third blood pressure value, measured by the measuring unit at a time of day different from the time of day of measurement of the first blood pressure value, and a fourth blood pressure value, included in second biological information at a time of day which is earlier, by a certain length of time, than the time of day when measurement of the third blood pressure value has been commenced, are further acquired. If a difference between the second blood pressure value and the fourth blood pressure value is greater than a difference between the first blood pressure value and the third blood pressure value, and if the difference between the second blood pressure value and the fourth blood pressure value exceeds a threshold value, it is determined that a failure is likely to be occurring in the sensing apparatus. Accordingly, it is possible to detect a failure in the sensing apparatus at an early stage, thus further increasing the period of time during which biological information obtained based on the pulse wave from the detector can be measured with high precision.

According to the twelfth aspect of the present invention, the first biological information is measured with higher precision than the second biological information obtained from the detector, and high-precision biological information is obtained from the measuring unit for calibration. This ensures the precision of the biological information obtained based on the pulse wave from the detector, enabling calculation of the biological information with high precision continuously in time.

According to the thirteenth aspect of the present invention, the detection unit detects the pulse wave beat by beat, and the first biological information and the second biological information are blood pressures. It is thereby possible for the biological information measuring apparatus to measure the blood pressure of each beat of the pulse wave continuously in time.

That is, according to each aspect of the present invention, it is possible to provide a biological information measuring apparatus which, through being worn constantly, is capable of acquiring accurate information while calibrating biological information continuously in time, and a method and a program using the same.

### BRIEF DESCRIPTION OF THE DRAWINGS

FIG. 1 is a block diagram illustrating a blood pressure measuring apparatus according to a first embodiment.

FIG. 2 is a diagram illustrating an example in which the blood pressure measuring apparatus of FIG. 1 is being worn on the wrist.

FIG. 3 is a diagram illustrating another example in which the blood pressure measuring apparatus of FIG. 1 is being worn on the wrist.

FIG. 4 is a diagram illustrating the time course of the cuff pressure and the pulse wave signal by oscillometric technique.

FIG. 5 is a diagram illustrating beat-by-beat changes in pulse pressure over time, and a pulse wave of one of the heartbeats.

FIG. 6 is a flowchart illustrating a calibration technique.

FIG. 7 is a flowchart for determining whether or not the capacity of the electric power source unit of the calibration device of FIG. 1 is low.

FIG. 8 is a block diagram illustrating a blood pressure measuring apparatus according to a second embodiment.

FIG. 9 is a flowchart for determining whether or not the number of measurements performed by the blood pressure measuring apparatus of the calibration device of FIG. 8 is large.

FIG. 10 is a block diagram illustrating a blood pressure measuring apparatus according to a third embodiment.

FIG. 11 is a flowchart for determining whether or not the fluctuation in blood pressure value of the sensing apparatus in FIG. 10 is large.

FIG. 12 is a flowchart for determining whether or not the difference in blood pressure value of the sensing apparatus in FIG. 10 is large.

FIG. 13 is a sequence diagram of the sensing apparatus and the calibration device, from activation of the sensing apparatus and the calibration device to continuous blood pressure measurement.

FIG. 14 is a sequence diagram of the sensing apparatus and the calibration device, from continuous blood pressure measurement to determination of recalibration.

### DETAILED DESCRIPTION

Hereinafter, a biological information measuring apparatus, and a method, and a program using the same according to embodiments of the present invention will be described with reference to the accompanying drawings. In the embodiments described below, components assigned with the same reference numbers are assumed to perform similar operations, and redundant descriptions thereof will be omitted.

The present embodiments have been made in response to the above-described circumstances, and aim to provide a biological information measuring apparatus which, through being worn constantly, is capable of acquiring accurate information while calibrating biological information continuously in time, and a method and a program using the same.

### First Embodiment

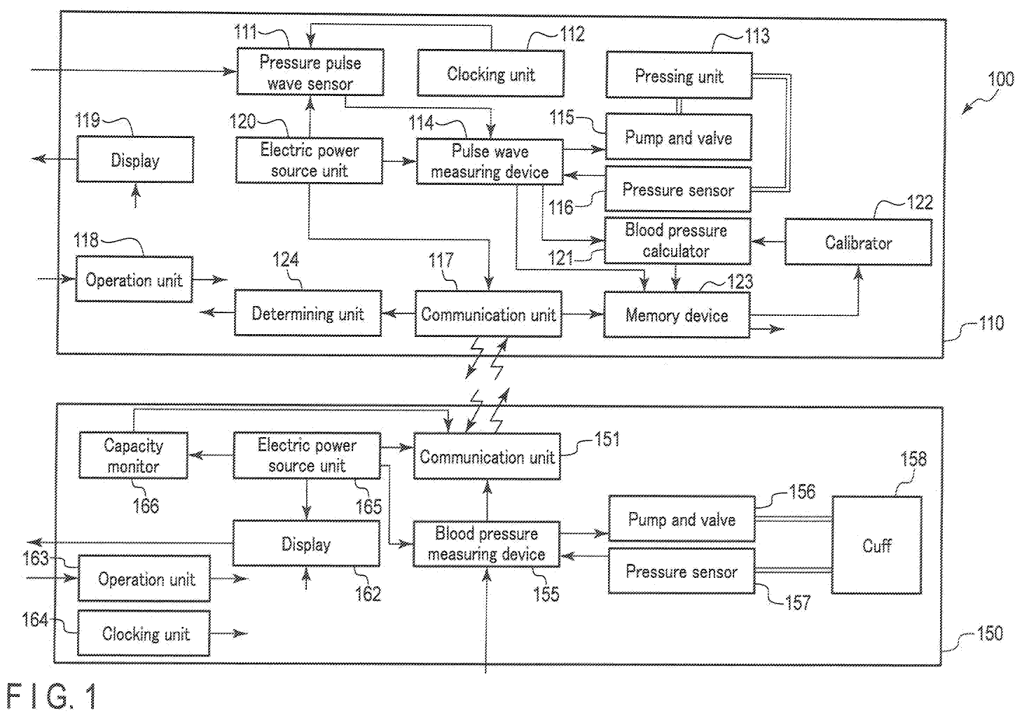

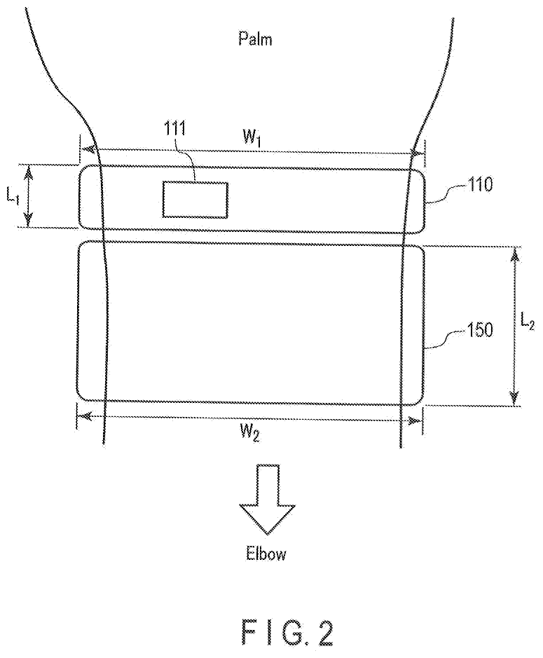

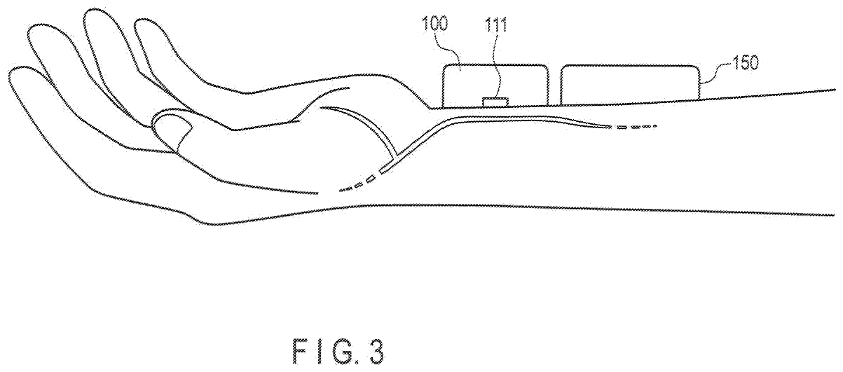

A blood pressure measuring apparatus 100 according to the present embodiment will be described, with reference to FIGS. 1, 2 and 3. FIG. 1 is a functional block diagram of the blood pressure measuring apparatus 100, illustrating details of a sensing apparatus 110 and a calibration device 150. FIG. 2 is a schematic perspective view, illustrating an example in which the blood pressure measuring apparatus 100 is being worn on the wrist, as seen from above the palm. A pressure pulse wave sensor 111 is disposed on the wrist side of the sensing apparatus 110. FIG. 3 is a schematic perspective view conceptually illustrating the blood pressure measuring apparatus 100 when being worn, as seen from the lateral side of the palm (i.e., the direction in which the fingers are aligned when the hand is open). FIG. 3 illustrates an example in which the pressure pulse wave sensor 111 is disposed orthogonal to the radial artery. It may appear from FIG. 3 that the blood pressure measuring apparatus 100 is simply laid on the palm side of the arm; however, the blood pressure measuring apparatus 100 is actually wrapped around the arm.

The blood pressure measuring apparatus 100 includes the sensing apparatus 110 and the calibration device 150. The sensing apparatus 110 includes the pressure pulse wave sensor 111, a clocking unit 112, a pressing unit 113, a pulse wave measuring device 114, a pump and valve 115, a pressure sensor 116, a communication unit 117, an operation unit 118, a display 119, an electric power source unit 120, a blood pressure calculator 121, a calibrator 122, a memory device 123, and a determining unit 124. The calibration device 150 includes a communication unit 151, a blood pressure measuring apparatus 155, a pump and valve 156, a pressure sensor 157, a cuff 158, a display 162, an operation unit 163, a clocking unit 164, an electric power source unit 165, and a capacity monitor 166.

The blood pressure measuring apparatus 100 is circular, wrapped around the wrist, etc. like a bracelet, and measures the blood pressure based on biological information. The sensing apparatus 110 is disposed on a side of the wrist closer to the palm than the calibration device 150, as shown in FIGS. 2 and 3. In other words, the sensing apparatus 110 is disposed farther from the elbow than the calibration device 150. In the present embodiment, the sensing apparatus 110 is disposed in such a manner that the pressure pulse wave sensor 111 is positioned above the radial artery, and, in accordance with this disposition, the calibration device 150 is disposed on the side closer to the elbow than the sensing apparatus 110. The sensing apparatus 110 and the calibration device 150 may be worn on different arms. It is generally preferable to dispose the sensing apparatus 110 and the calibration device 150 at the same height. It is further preferable to dispose the sensing apparatus 110 and the calibration device 150 at the height of the heart.

A length L₁ of the sensing apparatus 110 is set to be smaller than a length L₂ of the calibration device 150, as seen in the direction in which the arm extends. The length L₁ of the sensing apparatus 110 in the direction in which the arm extends is set to 40 mm or less, and more desirably, to 15 to 25 mm. A width W₁ of the sensing apparatus 110 is set to 4 to 5 cm, and a width W₂ of the calibration device 150 is set to 6 to 7 cm, as seen in the direction perpendicular to the direction in which the arm extends. In addition, the width W₁ and the width W₂ satisfy the relationship expressed as: 0 (or 0.5) cm<W₂−W₁<2 cm. Such a relationship prevents W₂ from being set too great, suppressing interference with the surroundings. By setting the size of the sensing apparatus 110 within such a range, the calibration device 150 is disposed on the side closer to the palm, thus facilitating detection of the pulse wave and keeping the precision in measurement. However, the calibration device 150 may be disposed on the upper arm during measurement.

The pressure pulse wave sensor 111 detects the pressure pulse wave continuously in time. For example, the pressure pulse wave sensor 111 detects the pressure pulse wave beat by beat. The pressure pulse wave sensor 111 is disposed on the palm side, as shown in FIG. 2, and is usually disposed parallel to the direction in which the arm extends, as shown in FIG. 3. The pressure pulse wave sensor 111 is capable of obtaining time-series data of the blood pressure value (blood pressure waveform), which changes according to the heartbeat.

The clocking unit 112 outputs time-of-day data to the pressure pulse wave sensor 111. The clocking unit 112 allows the pressure pulse wave sensor 111 to pass data on the pressure pulse wave, as well as the time-of-day data, to another component. The memory device 123 records, for example, the time-of-day data, as well as data to be stored therein.

The pressing unit 113 is an air bag that presses the sensor portion of the pressure pulse wave sensor 111 against the wrist, thereby increasing the sensitivity of the sensor.

The pulse wave measuring device 114 receives the pressure pulse wave data, as well as the time-of-day data, from the pressure pulse wave sensor 111, and passes the received data to the blood pressure calculator 121 and the memory device 123. The pulse wave measuring device 114 controls the pump and valve 115 and the pressure sensor 116 to pressurize or depressurize the pressing unit 113, and adjusts the pressure pulse wave sensor 111 so as to be pressed against the radial artery at the wrist.

The communication unit 117 and the communication unit 151 communicate with each other by a communication system that enables short-distance data exchange. Examples of the communication system used by these communication units include a short-distance wireless communication system, such as Bluetooth (registered trademark), TransferJet (registered trademark), ZigBee (registered trademark), and IrDA (registered trademark).

The pump and valve 115 pressurizes or depressurizes the pressing unit 113, according to an instruction from the pulse wave measuring device 114. The pressure sensor 116 monitors the pressure of the pressing unit 113, and notifies the pulse wave measuring device 114 of the pressure value of the pressing unit 113.

The electric power source unit 120 supplies electric power to each component of the sensing apparatus 110.

The blood pressure measuring apparatus 155 measures the blood pressure, which is biological information, with higher precision than the pressure pulse wave sensor 111. The blood pressure measuring apparatus 155 measures, for example, the blood pressure intermittently, not continuously in time, and passes the measured values to the memory device 123 and the calibrator 122 via the communication units 151 and 117. The blood pressure measuring apparatus 155 measures the blood pressure using, for example, oscillometric technique. Moreover, the blood pressure measuring apparatus 155 controls the pump and valve 156 and the pressure sensor 157 to pressurize or depressurize the cuff 158, thereby measuring the blood pressure. The blood pressure measuring apparatus 155 passes data on the systolic blood pressure, as well as the time of day of measurement thereof, and data on the diastolic blood pressure, as well as the time of day of measurement thereof, to the memory device 123, via the communication units 151 and 117. The systolic blood pressure is also referred to as SBP, while the diastolic blood pressure is also referred to as DBP.

The memory device 123 sequentially acquires and stores data on the pressure pulse wave from the pulse wave measuring device 114, as well as the time of day of detection thereof, and acquires and stores data on the SBP and the DBP from the blood pressure measuring apparatus 155, as well as the times of day of measurement thereof, at the time of operation of the blood pressure measuring apparatus 155, via the communication units 151 and 117. Also, the memory device 123 records, in association with the measured biological information, model information and/or unique identification information of the calibration device, in which first biological information for calibration is measured (by the blood pressure measuring apparatus 155), which is used to calculate the measured biological information (continuous blood pressures). It is thereby possible to know, from the measured biological information, which blood pressure monitor (model, unique device number, etc.) has been used for the calibration.

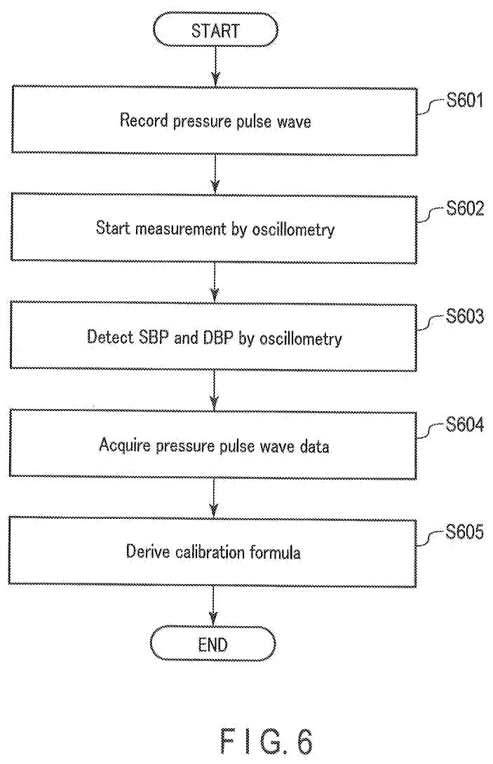

The calibrator 122 acquires, from the memory device 123, the data on the SBP and DBP, measured by the blood pressure measuring apparatus 155, as well as the time of day of measurement thereof, and the data on the pressure pulse wave measured by the pulse wave measuring device 114 of the sensing apparatus 110, as well as the time of day of measurement thereof. The calibrator 122 calibrates the pressure pulse wave from the pulse wave measuring device 114, based on the blood pressure value from the blood pressure measuring apparatus 155. Of several calibration techniques that may be adopted by the calibrator 122, an example calibration technique will be described later in detail, with reference to FIG. 6.

The blood pressure calculator 121 receives a calibration technique from the calibrator 122, calibrates the pressure pulse wave data from the pulse wave measuring device 114, and stores the blood pressure data obtained from the pressure pulse wave data in the memory device 123, as well as the time-of-day-of-measurement data.

An electric power source unit 165 supplies electric power to each component of the calibration device 150.

The display 162 displays various types of information, such as the results of the blood pressure measurement, to the user. The display 162 receives data from, for example, the blood pressure measuring apparatus 155, and displays the contents of the received data. For example, the display 162 displays the blood pressure value data, as well as the time-of-day-of-measurement data.

The display 119 also displays various types of information, such as the results of the blood pressure measurement, to the user. The display 119 receives data from, for example, the pulse wave measuring device 114, and displays the contents of the received data. For example, the display 119 displays the pressure pulse wave data, as well as the time-of-day-of-measurement data.

The operation unit 163 receives an operation from the user. The operation unit 163 includes, for example, an operation button for causing the blood pressure measuring apparatus 155 to commence measurement, an operation button for performing calibration, and an operation button for initiating or terminating communication.

The operation unit 118 also receives an operation from the user. The operation unit 118 includes, for example, an operation button for causing the pulse wave measuring device 114 to commence measurement, and an operation button for initiating or terminating communication.

The clocking unit 164 generates time-of-day data and supplies the generated time-of-day data to a component that requires such data.

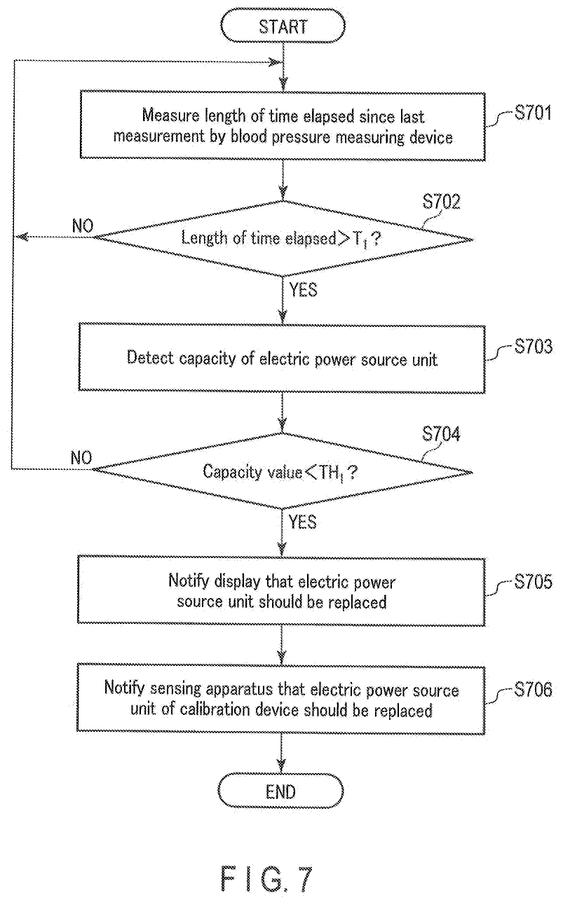

The capacity monitor 166 monitors the capacity of the electric power source unit 165 and transmits the monitored capacity to the sensing apparatus 110 via the communication units 151 and 117, and the determining unit 124 of the sensing apparatus 110 determines whether or not the calibration device 150 is still sufficiently capable of measuring and calibrating the blood pressure. Specifically, the capacity monitor 166 measures the capacity of the electric power source unit 165, and the determining unit 124 determines whether or not the capacity is smaller than the threshold value. Details of the operation of the capacity monitor 166 will be described later, with reference to FIG. 7.

At the time of implementation, a program for executing each of the above-described operations is stored in, for example, a secondary storage device included in each of the pulse wave measuring device 114, the calibrator 122, the blood pressure calculator 121, and the blood pressure measuring apparatus 155; and the central processing unit (CPU) executes a read operation of the stored program. The secondary storage device is, for example, a hard disk; however, it may be any device capable of storing data, such as a semiconductor memory, a magnetic memory device, an optical memory device, a magneto-optical disk, and a memory device employing the phase-change recording technology.

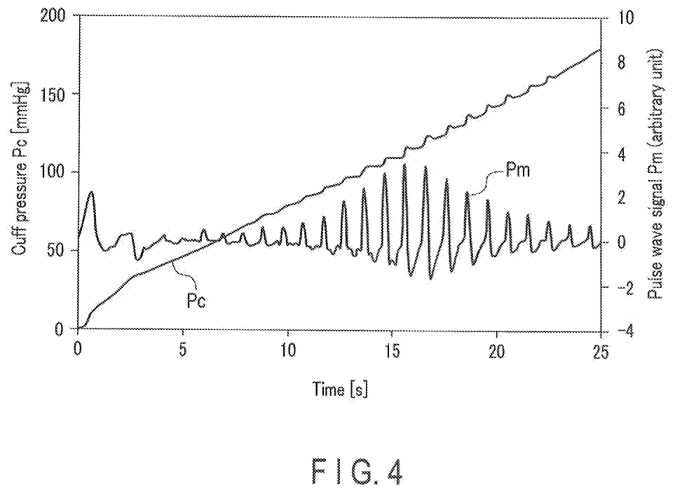

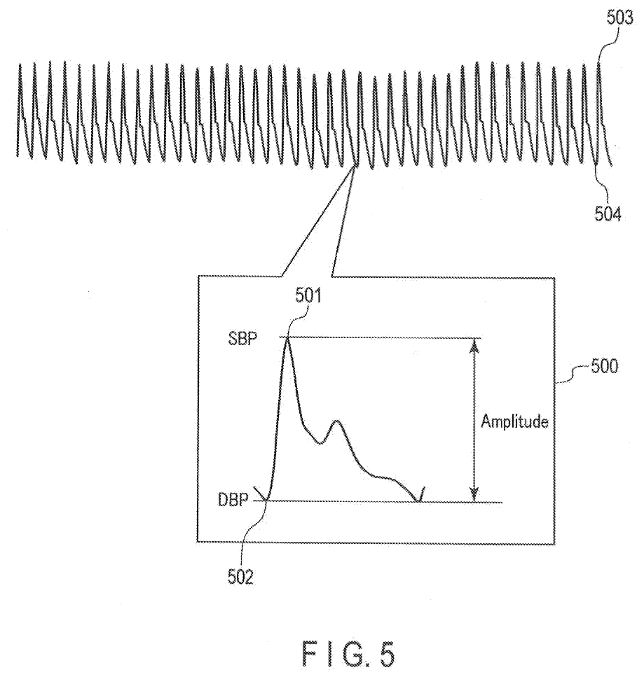

Next, operations performed by the pulse wave measuring device 114 and the blood pressure measuring apparatus 155 prior to calibration by the calibrator 122 will be described, with reference to FIGS. 4 and 5. FIG. 4 illustrates changes in cuff pressure and changes in magnitude of the pulse wave signal over time, during a blood pressure measurement by oscillometric technique. It can be seen from FIG. 4, illustrating changes in cuff pressure and changes in pulse wave signal over time, that the cuff pressure increases with time, and that the magnitude of the pulse wave signal gradually increases in tandem with the increase in the cuff pressure, and gradually decreases after reaching the maximum value. FIG. 5 illustrates time-series pulse pressure data acquired by beat-by-beat measurement of the pulse pressure. FIG. 5 also illustrates a waveform of a pressure pulse wave of one of the heartbeats.

A brief description will be given of the operation when the blood pressure measuring apparatus 155 performs a blood pressure measurement by oscillometric technique, with reference to FIG. 4. The blood pressure value may be calculated not only in the course of pressurization, but also in the course of depressurization; however, only the course of pressurization is illustrated as an example.

When the user instructs a blood pressure measurement by oscillometric technique via the operation unit 163 provided in the calibration device 150, the blood pressure measuring apparatus 155 commences operation and initializes the memory area for processing. Moreover, the blood pressure measuring apparatus 155 deactivates the pump of the pump and valve 156 to open the valve, and allows the air in the cuff 158 to be discharged. Subsequently, control is performed to set the output value of the pressure sensor 157 at that point in time as a value corresponding to the atmospheric pressure (adjusted to 0 mmHg).

Subsequently, the blood pressure measuring apparatus 155 functions as a pressure controller, and performs control to deliver air to the cuff 158 by closing the valve of the pump and valve 156, and then driving the pump. This expands the cuff 158, and gradually increases the cuff pressure (Pc in FIG. 4) for pressurization. To calculate blood pressure values in the course of pressurization, the blood pressure measuring apparatus 155 monitors the cuff pressure Pc using the pressure sensor 157, and acquires, as the pulse wave signal Pm as shown in FIG. 4, the fluctuation component of the arterial volume generated in the radial artery at the wrist, which is the measurement site.

Thereafter, the blood pressure measuring apparatus 155 attempts to calculate the blood pressure values (SBP and DBP) based on the pulse wave signal Pm acquired at that point in time, by applying a known algorithm using oscillometric technique. If the blood pressure values cannot yet be calculated at this point in time due to shortage of data, a pressurization treatment similar to the above-described is repeated, unless the cuff pressure Pc reaches the upper-limit pressure (which is preset to, for example, 300 mmHg for safety purposes).

After the blood pressure values are thus calculated, the blood pressure measuring apparatus 155 performs control to discharge the air in the cuff 158 by deactivating the pump of the pump and valve 156 so as to open the valve. Lastly, the results of measurement of the blood pressure values are passed to the calibrator.

Next, a description will be given of the beat-by-beat measurement of the pulse wave by the pulse wave measuring device 114, with reference to FIG. 5. The pulse wave measuring device 114 measures the pulse wave using, for example, tonometry.

In order for the pressure pulse wave sensor 111 to realize the optimum measurement, the pulse wave measuring device 114 controls the pump and valve 115 and the pressure sensor 116 to reach a predetermined optimum pressing force, by increasing the internal pressure of the pressing unit 113 to the optimum pressing force and keeping the optimum pressing force. Next, when the pressure pulse wave is detected by the pressure pulse wave sensor 111, the pulse wave measuring device 114 acquires the detected pressure pulse wave.

The pressure pulse wave is continuously detected beat by beat as a waveform as shown in FIG. 5. The pressure pulse wave 500 in FIG. 5 represents a pressure pulse wave of one beat, with a pressure value 501 corresponding to the SBP and a pressure value 502 corresponding to the DBP. Normally, the SBP 503 and the DBP 504 fluctuate according to the heartbeat of the pressure pulse, as shown by the time-series pressure pulse wave in FIG. 5.

Next, a description will be given of the operation of the calibrator 122, with reference to FIG. 6.

The calibrator 122 calibrates the pressure pulse wave detected by the pulse wave measuring device 114, using the blood pressure value measured by the blood pressure measuring apparatus 155. That is, the calibrator 122 determines the maximum value 501 and the minimum value 502 of the blood pressure values of the pressure pulse wave detected by the pulse wave measuring device 114.

(Calibration Technique)

The pulse wave measuring device 114 commences recording data on the pressure pulse wave, as well as the time of day of measurement thereof, and sequentially stores the pressure pulse wave data in the memory device 123 (step S601). Thereafter, measurement by oscillometric technique is commenced by, for example, a user activating the blood pressure measuring apparatus 155 by using the operation unit 163 (step S602). The blood pressure measuring apparatus 155 records SBP data and DBP data, as well as the times of day of detection of the SBP and DBP by oscillometric technique, based on the pulse wave signal Pm, and stores the recorded SBP data and DBP data in the memory device 123 (step S603).

The calibrator 122 acquires a pressure pulse wave corresponding to the SBP data and the DBP data from the pressure pulse wave data (step S604). The calibrator 122 derives a calibration formula based on the maximum value 501 of the pressure pulse wave corresponding to the SBP and the minimum value 502 of the pressure pulse wave corresponding to the DBP (step S605).

Next, monitoring of the battery capacity of the calibration device 150 of the blood pressure measuring apparatus 100 according to the present embodiment will be described, with reference to FIG. 7.

The capacity monitor 166 measures a length of time that has elapsed since the last measurement performed by the blood pressure measuring apparatus 155 of the calibration device 150 (step S701). The capacity monitor 166 determines, at a certain time interval, whether or not the length of time elapsed is greater than a preset length of time T₁ (step S702). If the length of time elapsed is not greater than T₁, the processing returns to step S701; if greater, then the processing advances to step S703. In step S703, the capacity monitor 166 detects the capacity of the electric power source unit 165. Thereafter, upon receiving the capacity of the electric power source unit 165 via the communication units 151 and 117, the determining unit 124 determines whether or not the capacity detected by the capacity monitor 166 in step S703 is smaller than a preset threshold value TH₁ (step S704). If the detected capacity is not smaller than TH₁, the processing returns to step S701; if smaller, then the processing advances to step S705. In step S705, the determining unit 124 controls the display 119 to display an indication that prompts replacement or charging of the electric power source unit 165. Moreover, the determining unit 124 notifies the calibration device 150, via the communication units 151 and 117, that the electric power source unit 165 of the calibration device 150 should be replaced or charged (step S706). Upon receiving the notification, the calibration device 150 may display, on the display 162, information that the electric power source unit 165 of the calibration device 150 should be replaced or charged. The displays 162 and 119 are not limited to displays, and may be prompters that prompt the user to make a certain action (replacement or charging in this example) by, for example, emitting a sound or causing haptically-appealing irregularities to occur on the surface of the apparatus. The above-described operations of the capacity monitor 166 and the determining unit 124 prevent the situation in which the calibration device 150 cannot perform calibration during continuous measurement and cannot perform accurate blood pressure measurement. It is thereby possible to keep performing continuous measurement of the blood pressure normally.

According to the first embodiment described above, since the sensing apparatus 110 and the calibration device 150 are separated, the necessity to align the calibration device 150 is reduced, and the pressure pulse wave sensor 111 of the sensing apparatus 110 can be disposed at the optimum position. Since the pulse wave is calibrated based on the first blood pressure value measured by the calibration device 150, the second blood pressure value is calculated based on the pulse wave, and the pulse wave is calculated based on the first blood pressure value measured by the calibration device 150, it is possible to calculate biological information with high precision based on the pulse wave, thus allowing the user to easily obtain high-precision biological information. Moreover, since the calibration device 150 is independently provided, the calibration device 150 can be mounted at a position appropriate for calibration with ease, regardless of the disposition of the sensing apparatus 110. Furthermore, the determining unit 124 performs determination as to whether or not the battery capacity of the calibration device 150 has decreased to a level at which the pulse wave cannot be calibrated, and if it is determined that calibration cannot be performed, the user is prompted to charge or replace the electric power source unit 165. This prevents the situation in which the calibration device 150 reaches the end of its life and cannot perform accurate calibration during, for example, continuous measurement. It is thereby possible to constantly perform calibration with a normal calibration value.

### Second Embodiment

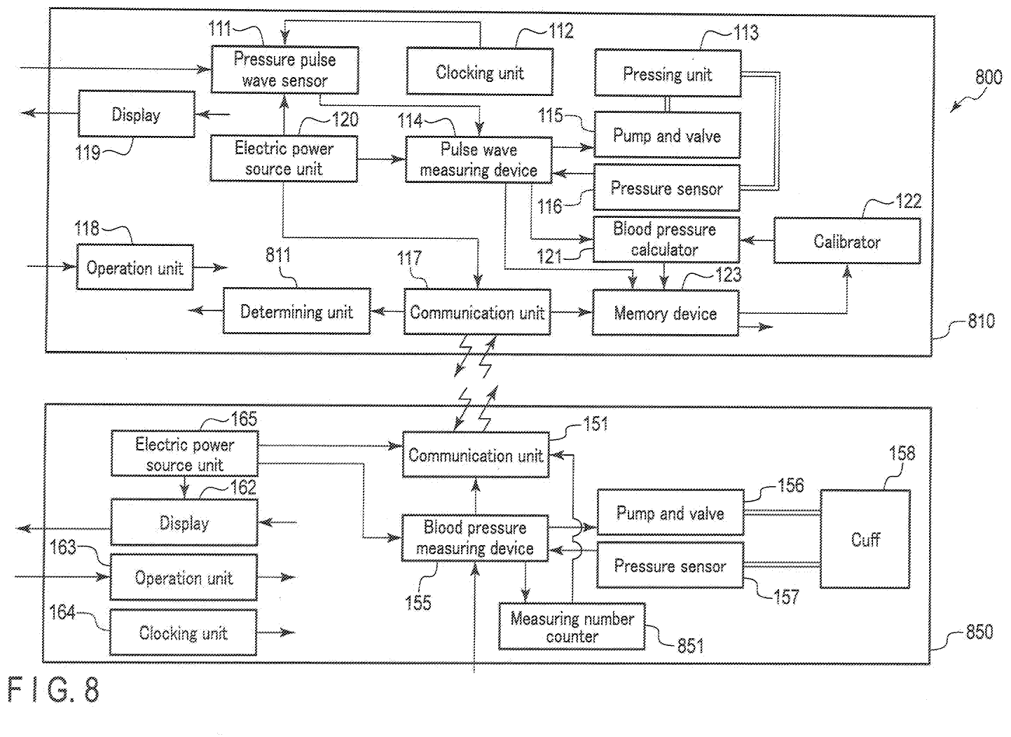

A blood pressure measuring apparatus 800 according to the present embodiment will be described, with reference to FIGS. 8, 2, and 3. FIG. 8 is a functional block diagram of the blood pressure measuring apparatus 800, illustrating details of a sensing apparatus 810 and a calibration device 850. The schematic perspective view of FIG. 2, illustrating an example in which the blood pressure measuring apparatus 100 is being worn on the wrist, as seen from above the palm, similarly applies to the blood pressure measuring apparatus 800. A pressure pulse wave sensor 111 is disposed on the wrist side of the sensing apparatus 110. The schematic perspective view of FIG. 3, conceptually illustrating the blood pressure measuring apparatus 100 when being worn, as seen from the lateral side of the palm (i.e., the direction in which the fingers are aligned when the hand is open), similarly applies to the blood pressure measuring apparatus 800. FIG. 3 illustrates an example in which the pressure pulse wave sensor 111 is disposed orthogonal to the radial artery. It may appear from FIG. 3 that the blood pressure measuring apparatus 100 is simply laid on the palm side of the arm; however, the blood pressure measuring apparatus 100 is actually wrapped around the arm. FIGS. 2 and 3 apply to the present embodiment, similarly to the first embodiment.

The blood pressure measuring apparatus 800 according to the present embodiment differs from the blood pressure measuring apparatus 100 according to the first embodiment in terms of the calibration device 850 and a determining unit 811 of the sensing apparatus 810.

The calibration device 850 and the sensing apparatus 810 of the present embodiment respectively correspond to the calibration device 150 of the first embodiment from which the capacity monitor 166 is removed and to which a measuring number counter 851 is added, and to the sensing apparatus 110 of the first embodiment from which the determining unit 124 is removed and to which the determining unit 811 is added. The measuring number counter 851 counts the number of, for example, SBPs and DBPs obtained during the blood pressure measurement performed by the blood pressure measuring apparatus 155. Alternatively, the counting may be performed by, for example, counting the number of times when the cuff is increased. It is only required that the items for counting be associated with the lifespan of the calibration device 850, and it is further preferable that such items be directly associated with the lifespan.

When the blood pressure measuring apparatus 155 uses oscillometric technique, the blood pressure values (e.g., the SBP and DBP) are measured in a single count, as described with reference to FIG. 4. The determining unit 811 determines whether or not the calibration device 850 is nearing the end of its life (or has already reached the end of its life) based on the number of measurements performed, and notifies the display 162 of the determination result. The determining unit 811 notifies the sensing apparatus 110 that the calibration device 850 is nearing the end of its life (or has already reached the end of its life). Upon receiving the notification, the sensing apparatus 110 causes the display 119 to display information that the calibration device 850 is nearing the end of its life (or has already reached the end of its life) to warn the user and prompt the user to replace the calibration device 850. Consequently, it is possible for the user to constantly use the calibration device 850, which functions normally, and to continuously detect the blood pressure with high precision.

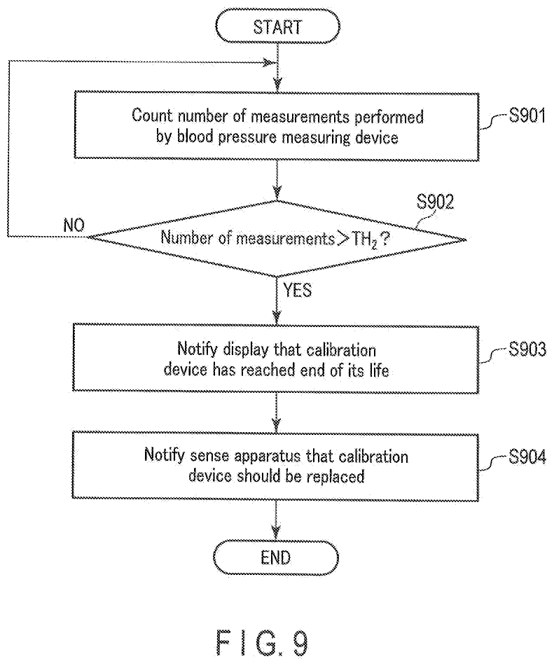

Next, a description will be made on the operations of the measuring number counter 851 and the determining unit 811, with reference to FIG. 9.

The measuring number counter 851 counts the number of measurements, representing the number of times when the blood pressure measuring apparatus 155 has measured the blood pressure values (hereinafter referred to as “SBP” and “DBP”) (step S901). In this example, let us assume that a single count corresponds to a measurement of the SBP and the DBP; however, a single count may be defined as a measurement of one of the SBP and the DBP. There are variations in how a single count is defined, and the threshold value (TH₂) for counting can be changed correspondingly.

Thereafter, determination is made as to whether or not the number of measurements counted by the measuring number counter 851 is greater than the threshold value TH₂. If the number of measurements is determined as being not greater than the threshold value TH₂, the processing returns to step S901; if greater, then the processing advances to step S903 (step S902). In step S903, the determining unit 811 notifies the display 162, via the communication units 117 and 151, that the calibration device 850 has reached the end of its life. Moreover, the determining unit 811 notifies the sensing apparatus 110 that the calibration device 850 should be replaced (step S904).

Upon receiving the notification, the sensing apparatus 110 may display, on the display 119, information that the calibration device 850 should be replaced. In step S903, a further instruction may be made by the determining unit 811 to deactivate the blood pressure measuring apparatus 155 by, for example, turning off its power supply. The displays 162 and 119 are not limited to displays, and may be prompters that prompt the user to make a certain action (replacement or charging in this example) by, for example, emitting a sound or causing haptically-appealing irregularities to occur on the surface of the apparatus.

The above-described operations of the measuring number counter 851 and the determining unit 811 prevent the situation in which the calibration device 850 cannot perform calibration during continuous measurement and cannot perform accurate blood pressure measurement. It is thereby possible to keep performing continuous measurement of the blood pressure normally.

According to the above-described second embodiment, the number of calibrations performed by the calibration device 850 is counted, and determination is made as to whether or not the number of calibrations performed has exceeded a certain number of times of usage. If the number of calibrations performed has exceeded a certain number of times of usage, the determining unit 811 determines that the calibration device 850 has reached the end of its life, and prompts replacement of the calibration device 850. It is thereby possible to prevent the situation in which the calibration device 850 reaches the end of its life and cannot perform calibration during, for example, continuous measurement, allowing calibration to be constantly performed with a normal calibration value, in addition to the advantageous effect of the first embodiment.

### Third Embodiment

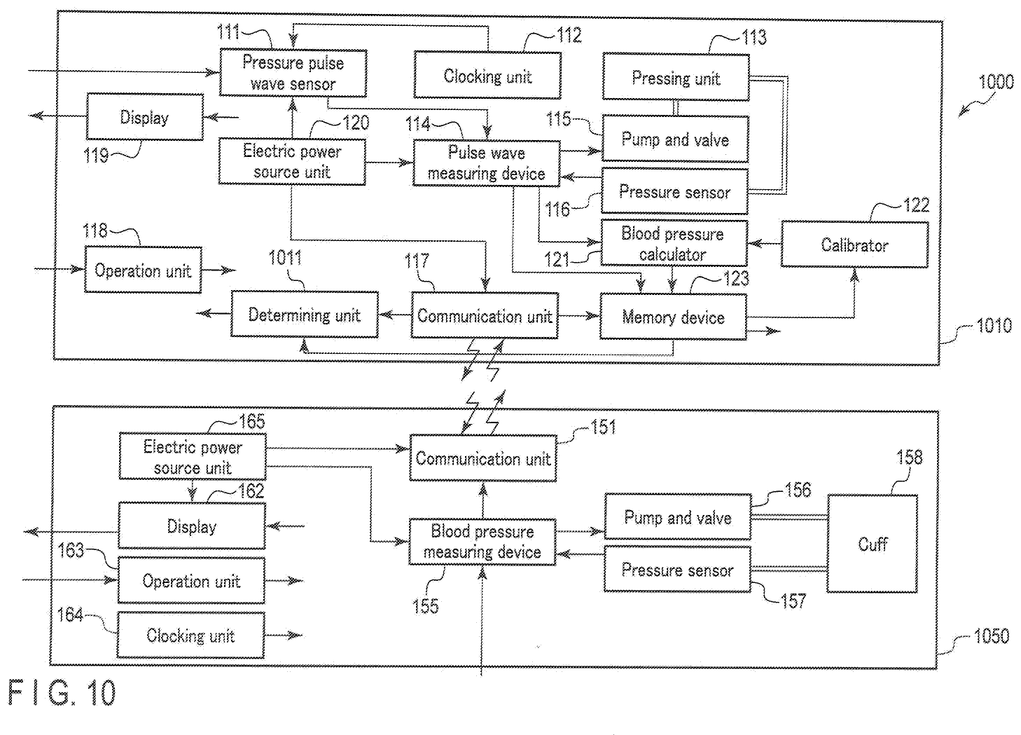

A blood pressure measuring apparatus 1000 according to the present embodiment will be described, with reference to FIGS. 10, 2, and 3. FIG. 10 is a functional block diagram of the blood pressure measuring apparatus 1000, illustrating details of a sensing apparatus 1010 and a calibration device 1050. The schematic perspective view of FIG. 2, illustrating an example in which the blood pressure measuring apparatus 100 is being worn on the wrist, as seen from above the palm, similarly applies to the blood pressure measuring apparatus 1000. A pressure pulse wave sensor 111 is disposed on the wrist side of the sensing apparatus 1010. The schematic perspective view of FIG. 3, conceptually illustrating the blood pressure measuring apparatus 100 when being worn, as seen from the lateral side of the palm (i.e., the direction in which the fingers are aligned when the hand is open), similarly applies to the blood pressure measuring apparatus 1000. FIG. 3 illustrates an example in which the pressure pulse wave sensor 111 is disposed orthogonal to the radial artery. It may appear from FIG. 3 that the blood pressure measuring apparatus 100 is simply laid on the palm side of the arm; however, the blood pressure measuring apparatus 100 is actually wrapped around the arm. FIGS. 2 and 3 apply to the present embodiment, similarly to the first embodiment.

The blood pressure measuring apparatus 1000 according to the present embodiment differs from the blood pressure measuring apparatus 100 of the first embodiment in that a determining unit 1011 is provided in the sensing apparatus 1010, and that a capacity monitor 166 is not provided in the calibration device 1050.

The calibration device 1050 and the sensing apparatus 1010 of the present embodiment respectively correspond to the calibration device 150 of the first embodiment from which the capacity monitor 166 is removed, and to the sensing apparatus 110 of the first embodiment from which the determining unit 124 is removed and to which the determining unit 1011 is added. The determining unit 1011 monitors a second blood pressure value (a blood pressure value based on the sensing apparatus 1010), which is stored in the memory device 123 and received from the pulse wave measuring device 114, and a first blood pressure value (blood pressure value measured by the calibration device 1050), which is stored in the memory device 123 via the communication units 151 and 117 and received from the blood pressure measuring apparatus 155, and determines, for example, the extent to which the difference between the first blood pressure value and the second blood pressure value is deviated from a certain threshold value. The second blood pressure value is a blood pressure value measured immediately before the measurement of the first blood pressure value. More accurately, the second blood pressure value is a blood pressure value measured by the pulse wave measuring device 114 at a time of day which is earlier, by a certain length of time, than the time of day when the blood pressure measuring apparatus 155 has commenced measurement. The second blood pressure value may be a mean value of blood pressure values measured during a certain period of time immediately before the measurement of the first blood pressure value. More accurately, the second blood pressure value may be a mean blood pressure value of blood pressure values measured by the pulse wave measuring device 114 during a certain period of time which is earlier, by a certain length of time, than the time of day when measurement of the first blood pressure value has been commenced by the blood pressure measuring apparatus 155.

A third blood pressure value measured prior to the measurement of the first blood pressure value may be used for comparison. In this case, a fourth blood pressure value measured by the pulse wave measuring device 114 is a blood pressure value measured immediately before the measurement of the third blood pressure value performed by the blood pressure measuring apparatus 155. Monitoring may be performed to determine whether or not the difference between the second blood pressure value and the fourth blood pressure value is greater than the difference between the first blood pressure value and the third blood pressure value, and the difference between the second blood pressure value and the fourth blood pressure value has exceeded a certain threshold value.

If the difference between the first blood pressure value and the second blood pressure value exceeds a certain threshold value, the determining unit 1011 determines that the second blood pressure value is abnormal, and that a failure is occurring in the sensing apparatus 1010, which is performing the measurement. If the difference between the second blood pressure value and the fourth blood pressure value is greater than the difference between the first blood pressure value and the third blood pressure value, and if the difference between the second blood pressure value and the fourth blood pressure value has exceeded a certain threshold value, the determining unit 1011 may determine that at least one of the second blood pressure value and the fourth blood pressure value is abnormal, and that a failure is occurring in the sensing apparatus 1010, which is performing the measurement.

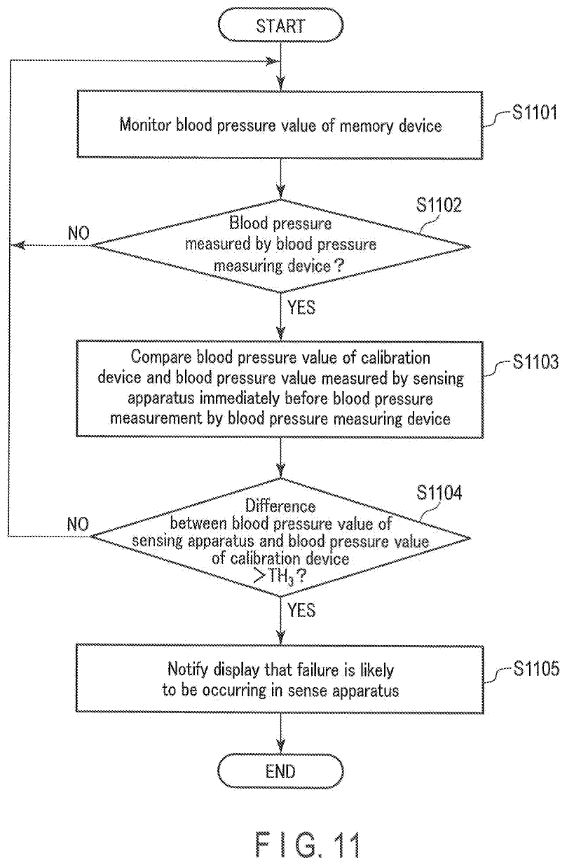

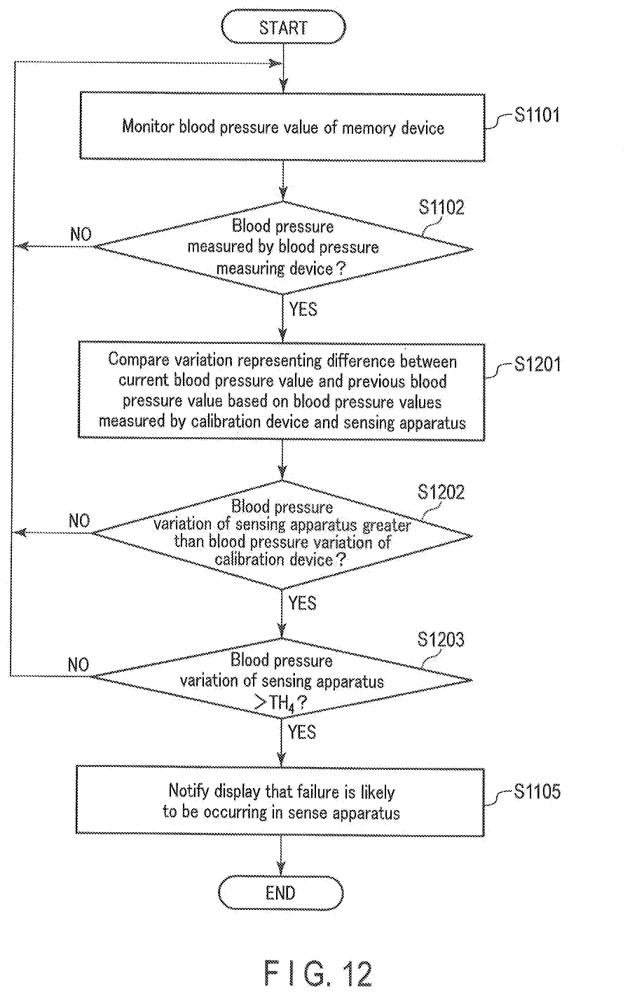

Next, a description will be given of the operation of the determining unit 1011, with reference to FIG. 11. Also, a description will be given of another example of the operation of the determining unit 1011, with reference to FIG. 12.

The determining unit 1011 monitors the first blood pressure value, which is a blood pressure value measured by the blood pressure measuring apparatus 155 of the calibration device 1050 and sequentially recorded in the memory device 123 (step S1101). The determining unit 1011 performs monitoring to determine whether or not the blood pressure measuring apparatus 155 has just measured the blood pressure. If it is determined that the blood pressure has not just been measured, the processing returns to step S1101, and if it is determined that the blood pressure has just been measured, the processing advances to step S1103 (step S1102). The second blood pressure value, which is a blood pressure value measured by the pulse wave measuring device 114 immediately before the measurement of the blood pressure by the blood pressure measuring apparatus 155, is acquired from the memory device 123, and is compared with the first blood pressure value measured by the blood pressure measuring apparatus 155 (step S1103). Determination is made as to whether or not the difference between the first blood pressure value and the second blood pressure value is greater than a predetermined threshold value TH₃. If the difference is determined as being greater, the processing advances to step S1105; if otherwise, then the processing returns to step S1101 (step S1104).

In step S1105, upon determining that a failure is likely to be occurring in the sensing apparatus 1010, the determining unit 1011 makes a notification to the display 162 via the communication units 151 and 117, and causes the display 162 to display information that a failure is likely to be occurring in the sensing apparatus 1010. In addition, the determining unit 1011 may notify the sensing apparatus 1010 that a failure is likely to be occurring in the sensing apparatus 1010. Upon receiving the notification, the display 119 of the sensing apparatus 1010 may display information that a failure is likely to be occurring in the sensing apparatus 1010. The displays 162 and 119 are not limited to displays, and may be prompters that prompt the user to make a certain action (replacement the sensing apparatus 1010 in this example) or notifiers that notify the user of information, by, for example, emitting a sound or causing haptically-appealing irregularities to occur on the surface of the apparatus.

Instead of comparing the second blood pressure value measured at a time of day immediately before the measurement of the blood pressure with the first blood pressure value, as in step S1103, a mean value of the second blood pressure values, measured during a certain period of time which is earlier, by a certain length of time, than the time of day when measurement has been commenced, may be compared with the first blood pressure value.

Next, another example of the operation of the determining unit 1011 will be described with reference to FIG. 12.

The processing until step S1102 is the same as that shown in FIG. 11. Thereafter, similarly to step S1103, a first blood pressure value and a second blood pressure value are acquired, and a third blood pressure value, measured by the blood pressure measuring apparatus 155 at a time of day different from the time of day of measurement of the first blood pressure value, and a fourth blood pressure value, measured by the pulse wave measuring device 114 at a time of day which is earlier by a certain length of time than the time of day when measurement of the third blood pressure value has been commenced, are acquired. The difference between the second blood pressure value and the fourth blood pressure value (also referred to as “blood pressure variation” of the sensing apparatus) measured by the pulse wave measuring device 114, and the difference between the first blood pressure value and the third blood pressure value (also referred to as a “blood pressure variation” of the calibration device) measured by the blood pressure measuring apparatus 155 are compared (step S1201).

Determination is made as to whether or not the difference between the second blood pressure value and the fourth blood pressure value is greater than a difference between the first blood pressure value and the third blood pressure value. If the difference is determined as being greater, the processing advances to step S1203; if otherwise, then the processing returns to step S1101 (step S1202). In step S1203, determination is made as to whether or not the difference between the second blood pressure value and the fourth blood pressure value, representing the blood pressure variation of the sensing apparatus, is greater than a preset threshold value TH₄. If the difference is determined as being greater, the processing advances to step S1105; if otherwise, the processing returns to step S1101.

The operation of the determining unit 1011 prevents the situation which sees the calibration device 150 is unable to perform calibration during continuous measurement and thus cannot perform accurate blood pressure measurement. It is thereby possible to keep performing continuous measurement of the blood pressure normally.

According to the third embodiment, determination is made as to whether or not the difference between the second blood pressure value of the sensing apparatus 1010 and the first blood pressure value of the calibration device 1050 is greater than a certain threshold value, based on the operation by the determining unit 1011. If the difference is determined as being greater, it is determined that a failure is likely to be occurring in the sensing apparatus 1010, and a notification is made to that effect. Thus, the sensing apparatus 1010 can be repaired or replaced immediately in the event of a failure. It is thereby possible to prevent the situation which sees measurement unable to be performed due to a failure in the sensing apparatus 1010 during, for example, continuous measurement, and to obtain a blood pressure value constantly calibrated with a normal calibration value.

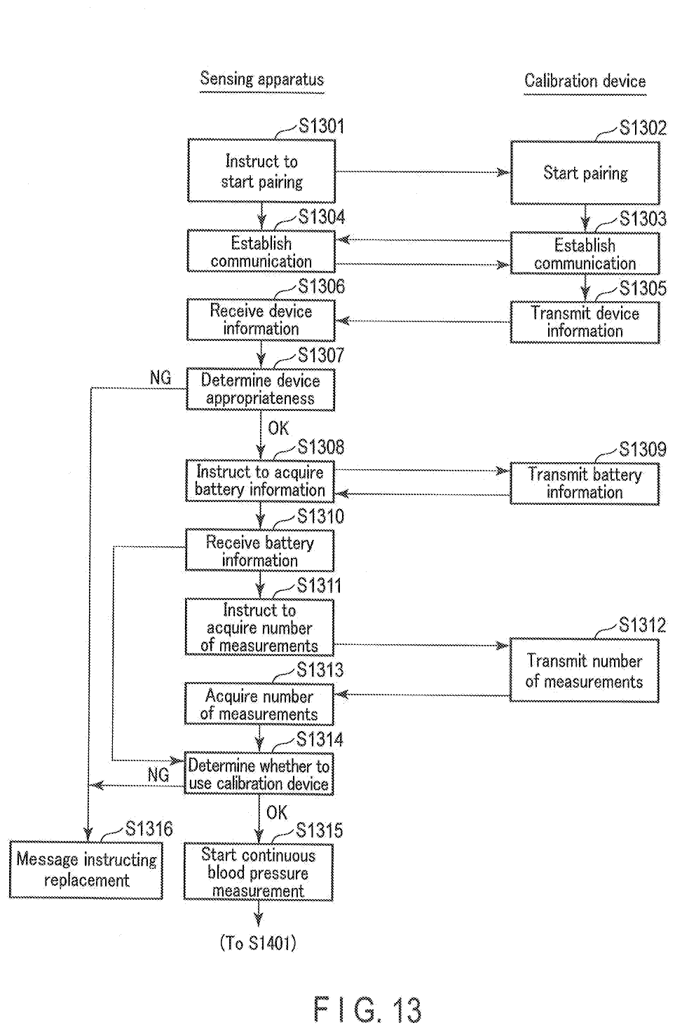

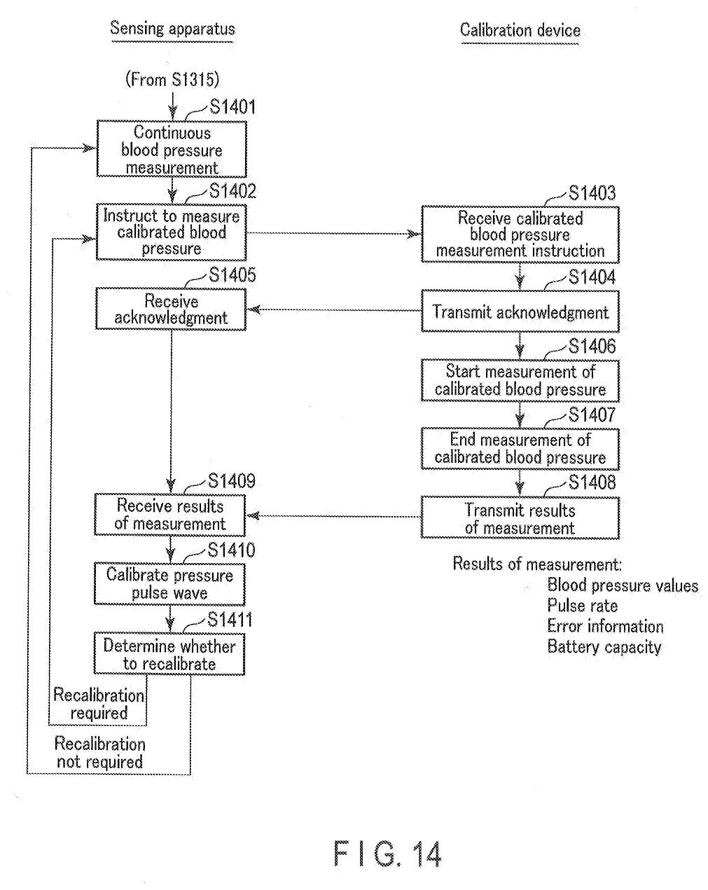

An example of the operation of the sensing apparatus and the calibration device in the case where all the embodiments are applied will be described. An example of a series of operations between the sensing apparatus having all the functions of the sensing apparatuses 110, 810, and 1010, and the calibration device having all the functions of the calibration devices 150, 850, and 1050 will be described, with reference to FIGS. 13 and 14.

The sensing apparatus instructs the calibration device to commence pairing with the calibration device (step S1301). The calibration device receives the instruction to commence pairing from the sensing apparatus, and commences pairing (step S1302). The calibration device establishes communication with the sensing apparatus as a result of the pairing (step S1303). Similarly, the sensing apparatus establishes communication with the calibration device as a result of pairing (step S1304).

The calibration device transmits device information of the calibration device itself after establishing communication with the sensing apparatus (step S1305). The device information includes specifications of the calibration device, such as the performance, the date of manufacturing, the type of communication system, and the version of the calibration device. The sensing apparatus receives device information (step S1306), and determines whether or not the calibration device is appropriate for the sensing apparatus (step S1307).

In step S1307, if it is determined that the calibration device is appropriate for the sensing apparatus, the processing advances to step S1308, and if it is determined that the calibration device is inappropriate, the processing advances to step S1316 and passes an replacement instruction message to the user.

Thereafter, the sensing apparatus instructs the calibration device to acquire battery information of the calibration device (step S1308). The calibration device receives the instruction from the sensing apparatus to transmit battery information, and transmits battery information of the calibration device itself to the sensing apparatus (step S1309). The sensing apparatus receives and acquires battery information of the calibration device (step S1310).

Thereafter, the sensing apparatus instructs the calibration device to acquire data on the number of measurements performed by the calibration device (step S1311). Upon receiving the instruction from the sensing apparatus to transmit data on the number of measurements, the calibration device transmits data on the number of measurements performed by the calibration device itself to the sensing apparatus (step S1312). The sensing apparatus receives and acquires the data on the number of measurements performed by the calibration device (step S1313).

The sensing apparatus determines whether or not the calibration device is to be used, based on the battery information acquired in step S1310 and the data on the number of measurements acquired in step S1313 (step S1314). The sensing apparatus determines whether or not the battery capacity is smaller than the threshold value TH₁, as in step S704, and determines whether or not the number of measurements is greater than the threshold value TH₂, as in step S902. In this case, if the battery capacity is greater than the threshold value TH₁, and the number of measurements is not greater than the threshold value TH₂, the calibration device is determined to be usable, and commences continuous blood pressure measurement (i.e., obtain time-series data of the blood pressure value, which changes according to the heartbeat) (step S1315). On the other hand, in cases other than the above-described case, namely, if the battery capacity is not greater than the threshold value TH₁ or if the number of measurements is greater than the threshold value TH₂, the calibration device is determined to be unusable, and a replacement instruction message promoting replacement of the calibration device is presented to the user (step S1316). The user replaces the calibration device with a new one, and commences operation from step S1301 between the sensing apparatus and the new calibration device. The above-described steps are repeated until step S1315.

At step S1315, the sensing apparatus commences continuous blood pressure measurement, and obtains time-series data of the blood pressure value, which changes according to the heartbeat (step S1401). The sensing apparatus instructs the calibration device to measure the calibrated blood pressure (step S1402). Upon receiving, from the sensing apparatus, the instruction to measure the calibrated blood pressure (step S), the calibration device transmits an acknowledgment indicating that the instruction has been received to the sensing apparatus (step S1404). The sensing apparatus receives the acknowledgment from the calibration device (step S1405). The sensing apparatus stands by until the results of measurement of the calibrated blood pressure are received from the calibration device.

On the other hand, the calibration device, instructed to measure the calibrated blood pressure, commences measuring the calibrated blood pressure (step S1406). After the calibration device completes measuring the calibrated blood pressure (step S1407), the results of measurement of the calibrated blood pressure are transmitted to the sensing apparatus (step S1408). The calibration device acquires, for example, the pulse rate, error information at the time of measurement, the battery capacity of the calibration device, and the number of times of calibration measurements, as well as the blood pressure value. Accordingly, the results of measurement include, for example, blood pressure values, the pulse rate, error information at the time of measurement, the battery capacity of the calibration device, and the number of measurements. Examples of the error information include failure to properly pressurize the cuff, movement of the arm or the body during blood pressure measurement, failure to properly detect the pulse wave, and other function abnormalities.

The sensing apparatus receives the results of measurement from the calibration device (step S1409), and then calibrates the pressure pulse wave based on the blood pressure value included in the results of measurement (step S1410). The results of measurement from the calibration device may be stored in the sensing apparatus, as well as data on the time of day of measurement of the result. Also, the blood pressure value acquired by calibrating the pressure pulse wave acquired in step S1410 may be stored in the sensing apparatus.

Thereafter, the sensing apparatus determines whether or not calibration needs to be performed again (step S1411). For example, the sensing apparatus compares the blood pressure value acquired by the calibration device in step S1408 and the blood pressure value measured by the pulse wave measuring device 114 immediately therebefore by continuous blood pressure measurement by the sensing apparatus (step S1401), as in step S1104. If the difference is greater than TH₃, the sensing apparatus determines that recalibration is required. If the difference is not greater than TH₃, it is determined that recalibration is not required, and the sensing apparatus keeps performing continuous blood pressure measurement (step S1401). If the difference is greater than TH₃, recalibration is not performed, and the processing advances to step S1316, and a message may be presented to the user indicating that a failure is likely to be occurring in the sensing apparatus, as in the example of FIG. 11.

Similarly, the sensing apparatus performs calculation based on the first blood pressure value acquired by the calibration device in step S1408, the second blood pressure value measured by the pulse wave measuring device 114 immediately therebefore by continuous blood pressure measurement by the sensing apparatus (step S1401), the third blood pressure value acquired by the calibration device in step S1408 at, for example, the last calibration, and the fourth blood pressure value acquired by the sensing apparatus immediately therebefore by continuous blood pressure measurement by the pulse wave measuring device 114 (step S1401), as in steps S1202 and S1203. Such calculation is performed in such a manner that |second blood pressure value-fourth blood pressure value|, which represents the variation in blood pressure value measured by the sensing apparatus, is greater than |first blood pressure value-third blood pressure value|, which represents the variation in blood pressure value measured by the calibration device (step S1202), and in the case of |second blood pressure value-fourth blood pressure value|>TH₄, it is determined that recalibration is required. Otherwise, it is determined that recalibration is not required, and the sensing apparatus keeps performing continuous blood pressure measurement (step S1401). If |second blood pressure value-fourth blood pressure value| is greater than |first blood pressure value-third blood pressure value| (step S1202), and in the case of |second blood pressure value-fourth blood pressure value|>TH₄, as in the example of FIG. 12, the processing may advance to step S1316 without performing recalibration, and a message may be presented to the user indicating that a failure is likely to be occurring in the sensing apparatus.

There are other examples in which the operation of the sensing apparatus changes according to the results of measurement. For example, if the sensing apparatus determines that the battery capacity included in the results of measurement is not sufficient to supply electric power to be consumed by the calibration device at the next calibration, the processing may advance to step S1316, and a message indicating that the battery of the calibration device should be replaced may be presented to the user. If the sensing apparatus determines that the battery cannot supply electric power to be consumed by the calibration device at the next calibration at a time of day at night that is regarded as a bedtime, the processing may advance to step S1401 and keep performing continuous blood pressure measurement, by not presenting the battery replacement message to the user and not performing calibration until morning after performing calibration of the pressure pulse wave in step S1410, since it is likely that the user is sleeping.

In the above-described embodiment, the pressure pulse wave sensor 111 detects, for example, the pressure pulse wave of the radial artery passing through the measurement site (e.g., the left wrist) (tonometric method). However, the configuration is not limited thereto. The pressure pulse wave sensor 111 may be configured to detect the pulse wave of the radial artery passing through a measurement site (e.g., the left wrist) as a change in impedance (impedance method). The pressure pulse wave sensor 111 may include a light-emitting element that emits light toward an artery passing through the corresponding portion of the measurement site, and a light-receiving element that receives reflected light (or transmitted light) of the emitted light, and may be configured to detect the pulse wave of the artery as changes in volume (photoelectric method). Moreover, the pressure pulse wave sensor 111 may include a piezoelectric sensor in contact with the measurement site, and may be configured to detect a strain caused by the pressure of the artery passing through the corresponding portion of the measurement site as a change in electric resistance (piezoelectric method). Furthermore, the pressure pulse wave sensor 111 may include a transmission element that transmits radio waves (transmission waves) toward an artery passing through the corresponding portion of the measurement site, and a reception element that receives reflection waves of the transmitted radio waves, and may be configured to detect a change in distance between the artery and the sensor caused by the pulse wave of the artery as a phase shift between the transmission waves and the reflection waves (radio wave irradiation method). In addition to the above-described methods, any method that enables observation of a physical quantity based on which the blood pressure can be calculated may be adopted.

In the above-described embodiment, the blood pressure measuring apparatuses 100, 800 and 1000 are assumed to be worn on the left wrist, which is the measurement site; however, the configuration is not limited thereto, and they may be worn on, for example, the right wrist. The measurement site is not limited to the wrist and may be any part through which an artery passes; examples include an upper limb such as an upper arm, and a lower limb such as an ankle and a thigh.

The apparatus of the present invention can also be realized by a computer and a program, and such a program may be recorded on a recording medium or provided through a network.

Moreover, the above-described apparatuses and their device portions can be implemented either as a hardware configuration or as a combined configuration of hardware resources and software. The software of the combined configuration may be a program pre-installed in a computer from a network or a computer-readable storage medium, to be executed by the processor of the computer to allow the computer to implement the functions of the respective apparatuses.

The present invention is not limited to the above-described embodiment and may be embodied in practice by modifying the structural elements without departing from the gist of the invention. In addition, various inventions can be made by suitably combining the structural elements disclosed in connection with the above-described embodiments. For example, some of the structural elements described in each of the embodiments may be deleted. Moreover, structural elements described in different embodiments may be suitably combined.

Furthermore, part or all of the above-described embodiments may be described as in the additional descriptions given below; however, the embodiments are not limited thereto.

(Additional Description 1)

A biological information measuring apparatus comprising a sensing apparatus including a first hardware processor and a calibration device including a second hardware processor and a memory,
the second hardware processor being configured to:
   intermittently measure first biological information; and
   transmit data including the first biological information to the sensing apparatus,
the first hardware processor is configured to:
   detect a pulse wave continuously in time;
   receive the data from the calibration device;
   calibrate the pulse wave based on the first biological information, and calculate second biological information from the pulse wave, and
wherein the memory includes
   a memory device that stores the second biological information.

(Additional Description 2)

A method of measuring biological information comprising:

intermittently measuring first biological information using at least one hardware processor; and

transmitting data including the first biological information to the sensing apparatus using said at least one hardware processor; and

detecting a pulse wave continuously in time using said at least one hardware processor;

transmitting data including the pulse wave to the calibration device using said at least one hardware processor; and

calibrating the pulse wave based on the first biological information and calculating second biological information from the pulse wave, using at least one hardware processor.

What is claimed is:

1. A biological information measuring apparatus comprising a sensing apparatus and a calibration device, wherein
the calibration device comprises:
a measuring device configured to intermittently measure first biological information; and
a transmitter configured to transmit data including the first biological information to the sensing apparatus,
the sensing apparatus comprises:
a detector configured to detect a pulse wave continuously in time;
a receiver configured to receive the data from the calibration device; and
a calculator configured to calibrate the pulse wave based on the first biological information, and calculate second biological information based on the pulse wave, and
the detector and the measuring unit are configured to be provided at an identical site.

2. A biological information measuring apparatus comprising a sensing apparatus and a calibration device, wherein
the calibration device comprises:
a measuring device configured to intermittently measure first biological information; and
a transmitter configured to transmit data including the first biological information to the sensing apparatus, and
the sensing apparatus comprises:
a detector configured to detect a pulse wave continuously in time;
a receiver configured to receive the data from the calibration device;
a calculator configured to calibrate the pulse wave based on the first biological information, and calculate second biological information based on the pulse wave; and
an instruction transmitter configured to transmit an instruction to measure the first biological information to the calibration device.

3. The apparatus according to claim 1, wherein the detector is configured to be disposed on a wrist of a living body, and the measuring unit is disposed closer to an upper arm than the detector.

4. The apparatus according to claim 2, wherein the detector and the measuring unit are configured to be provided at an identical site.

5. The apparatus according to claim 1, wherein the calibration device further comprises:
an electric power source unit configured to supply electric power to an internal device portion; and
a monitor configured to monitor a battery capacity of the electric power source unit,
the transmitter is configured to transmit capacity data including the battery capacity to the sensing apparatus upon completion of measurement by the measuring device or upon activation of the calibration device,
the receiver is configured to receive the capacity data, and
the sensing apparatus comprises:
a capacity-determining unit configured to determine, based on the capacity data, whether or not the battery capacity has decreased to a level at which the pulse wave cannot be calibrated.

6. The apparatus according to claim 5, further comprising: a prompter configured to prompt charging or replacement of the electric power source unit upon determining that the capacity-determining unit fails to perform calibration.

7. The apparatus according to claim 1, wherein the calibration device further comprises a counting unit configured to count a number of measurements performed by the measuring unit,
the transmitter is configured to transmit number data containing the number of measurements performed to the sensing apparatus upon completion of measurement by the measuring device or upon activation of the calibration device,
the receiver is configured to receive the number data, and
the sensing apparatus comprises a number-determining unit configured to determine, based on the number data, whether or not the number of measurements performed has exceeded a certain number of times of usage.

8. The apparatus according to claim 7, further comprising a prompter configured to prompt replacement of the calibration device if the number of measurements performed has exceeded a certain number of times of usage.

9. A biological information measuring apparatus comprising a sensing apparatus and a calibration device, wherein
the calibration device comprises:
a measuring device configured to intermittently measure first biological information; and
a transmitter configured to transmit data including the first biological information to the sensing apparatus, and
the sensing apparatus comprises:
a detector configured to detect a pulse wave continuously in time;
a receiver configured to receive the data from the calibration device;
a calculator configured to calibrate the pulse wave based on the first biological information, and calculate second biological information based on the pulse wave;
an acquisition unit configured to acquire a first blood pressure value included in the first biological information and a second blood pressure value included in second biological information at a time of day which is earlier, by a certain length of time, than a time of day when the measuring unit has commenced measurement; and
a failure-determining unit configured to determine that a failure is likely to be occurring in the sensing apparatus if a difference between the first blood pressure value and the second blood pressure value is equal to or greater than a threshold value.

10. The apparatus according to claim 1, further comprising:
an acquisition unit configured to acquire a first blood pressure value included in the first biological information and a mean blood pressure value of a second blood pressure value included in second biological information during a certain period of time which is earlier, by a certain length of time, than a time of day when the measuring unit has commenced measurement; and
a failure-determining unit configured to determine that a failure is likely to be occurring in the sensing apparatus if a difference between the first blood pressure value and the mean blood pressure value is equal to or greater than a threshold value.

11. A biological information measuring apparatus comprising a sensing apparatus and a calibration device, wherein
the calibration device comprises:
a measuring device configured to intermittently measure first biological information; and
a transmitter configured to transmit data including the first biological information to the sensing apparatus, and
the sensing apparatus comprises:
a detector configured to detect a pulse wave continuously in time;
a receiver configured to receive the data from the calibration device;
a calculator configured to calibrate the pulse wave based on the first biological information, and calculate second biological information based on the pulse wave;
an acquisition unit configured to acquire a first blood pressure value included in the first biological information, and a second blood pressure value included in second biological information at a time of day which is earlier, by a certain length of time, than a time of day when measurement of the first blood pressure value has been commenced, and further acquire a third blood pressure value measured by the measuring unit at a time of day different from the time of day of measurement of the first blood pressure value, and a fourth blood pressure value included in second biological information at a time of day which is earlier, by a certain length of time, than the time of day when measurement of the third blood pressure value has been commenced; and
a failure-determining unit configured to determine that a failure is likely to be occurring in the sensing apparatus if a difference between the second blood pressure value and the fourth blood pressure value is greater than a difference between the first blood pressure value and the third blood pressure value, and if the difference between the second blood pressure value and the fourth blood pressure value exceeds a threshold value.

12. The apparatus according to claim 1, wherein the measuring unit is configured to measure the first biological information with higher precision than second biological information obtained from the detector.

13. The apparatus according to claim 1, wherein the detector is configured to detect the pulse wave beat by beat, and the first biological information and the second biological information are blood pressures.

14. A method of measuring biological information in a biological information measuring apparatus comprising a sensing apparatus configured to detect a pulse wave and a calibration device configured to measure first biological information, the method comprising:
in the calibration device:
intermittently measuring first biological information; and
transmitting data including the first biological information to the sensing apparatus; and
in the sensing apparatus:
detecting a pulse wave continuously in time;
receiving the data from the calibration device;
calibrating the pulse wave based on the first biological information; and
calculating second biological information from the pulse wave, wherein a portion that detects the pulse wave continuously in time and a portion that intermittently measures the first biological information are both provided at an identical site.

15. A non-transitory computer readable medium storing a computer program which is executed by a computer to provide the steps of:
intermittently measuring first biological information;
transmitting data including the first biological information to the sensing apparatus;
detecting a pulse wave continuously in time;
receiving the data from the calibration device;
calibrating the pulse wave based on the first biological information; and
calculating second biological information from the pulse wave, a portion that detects the pulse wave and a portion that intermittently measures the first biological information being both provided at an identical site.
